(19) United States
(12) Patent Application Publication
(10) Pub. No.: US 2021/0181159 A1
(43) Pub. Date: Jun. 17, 2021

(54) SYSTEMS TO MONITOR CHARACTERISTICS OF MATERIALS INVOLVING OPTICAL AND ACOUSTIC TECHNIQUES

(71) Applicant: IDEACURIA INC., Richmond Hill (CA)

(72) Inventor: Gregory A. AGOSTINELLI, Richmond Hill (CA)

(21) Appl. No.: 16/760,865

(22) PCT Filed: Nov. 1, 2018
(86) PCT No.: PCT/IB2018/058589

§ 371 Date: Apr. 30, 2020

Related U.S. Application Data
(60) Provisional application No. 62/581,487, filed on Nov. 3, 2017.
(60) Provisional application No. 62/619,900, filed on Jan. 21, 2018.

(51) Int. Cl. G01N 29/24; G01N 29/46; G01N 29/44

(52) U.S. Cl. CPC G01N 29/2437; G01N 29/46; G01N 33/02; G01N 2291/022; G01N 29/4472

(57) ABSTRACT

An example system for monitoring a characteristic of a material. The system includes a stimulator to provide a stimulus signal to the material. The stimulus signal includes at least one of an electrical signal, a magnetic signal, an optical signal, and an acoustic signal. The system includes a sensor to measure a response signal from the material. The response signal includes at least one of an electrical signal, a magnetic signal, an optical signal, and an acoustic signal. At least one of the stimulus signal and the response signal includes an optical signal or an acoustic signal. The system further includes a controller in communication with the stimulator and the sensor to apply machine learning to determine a characteristic of the material based on the stimulus signal and the response signal, wherein the characteristic is not directly measurable.

### CROSS-REFERENCE TO RELATED APPLICATIONS

This application claims priority to U.S. 62/581,487, filed Nov. 3, 2017, and to U.S. 62/619,900, filed Jan. 21, 2018, each of which are incorporated herein by reference.

### FIELD

The present disclosure relates generally to material monitoring.

### BACKGROUND

Many materials have characteristics that change over time. These materials may be part of products such as foods, beverages, and chemical products. These products may have the potential to expire or become contaminated, but users of such products generally do not have a reliable means of non-invasively monitoring the status of the characteristics of these products.

For example, many beverages and foodstuff are provided with an estimated “best before” date which serves as a crude benchmark for estimating when a product has spoiled or passed its ideal consumption point, and the typical consumer is expected to trust that the product contained within the packaging is still in good condition upon consumption. As another example, certain beverages, such as wines, are known to change flavor characteristics in desirable ways over time, and it may be desirable to consume such products during a particular window of time. As another example, drinking water from a municipal water distribution system may become contaminated in transit to the end-user's household, long after having been cleansed by a water treatment facility. As yet another example, chemical products may react over time or undergo physical changes spontaneously over time or due to changing environmental factors. As yet another example, another material which may be monitored is a person's biological waste for indications of the person's health.

Current solutions to monitoring the characteristics of such materials typically involve invasive testing of the product, which may be destructive. Many solutions require that the container be opened, thus altering the product's environment, and in many cases accelerating a degradation process.

### SUMMARY

According to an aspect of the specification, a system for monitoring a characteristic of a material is provided. The system includes a stimulator to provide a stimulus signal to the material, the stimulus signal including at least one of an electrical signal, a magnetic signal, an optical signal, and an acoustic signal. The system further includes a sensor to measure a response signal from the material, the response signal including at least one of an electrical signal, a magnetic signal, an optical signal, and an acoustic signal. At least one of the stimulus signal and the response signal may include an optical signal or an acoustic signal. Further, the stimulus signal may include an electrical signal, a magnetic signal, an optical signal, and an acoustic signal, and the response signal may include an electrical signal, a magnetic signal, an optical signal, and an acoustic signal. The system further includes a controller in communication with the stimulator and the sensor. The controller is to apply machine learning to determine a characteristic of the material based on the stimulus signal and the response signal, wherein the characteristic is not directly measurable. The machine learning is applied via a machine learning model trained with library data of previously measured stimulus signals and response signals to recognize the characteristic of the material.

According to another aspect of the specification, a method for monitoring a characteristic of a material is provided. The method involves providing a stimulus signal to a material, the stimulus signal including at least one of an electrical signal, a magnetic signal, an optical signal, and an acoustic signal. The method further involves measuring a response signal from the material, the response signal including at least one of an electrical signal, a magnetic signal, an optical signal, and an acoustic signal. At least one of the stimulus signal and the response signal includes an optical signal or an acoustic signal. The method further involves determining a characteristic of the material based on the stimulus signal and the response signal, wherein the characteristic is not directly measurable, by applying machine learning via a machine learning model trained with library data of previously measured stimulus signals and response signals to recognize the characteristic of the material. The method may be performed by any of the systems and/or devices described herein. Further, the method may be instantiated in a non-transitory computer-readable medium which, when executed, causes a processor of a computing device to perform the method.

In some examples, the stimulus signal may include an electrical signal, a magnetic signal, an optical signal, and an acoustic signal, and the response signal may include an electrical signal, a magnetic signal, an optical signal, and an acoustic signal.

In some examples, the method may involve receiving an indication of the stimulus signal from a controller via the network and transmitting the indication of the stimulus signal to a stimulator via an electrical circuit. The method may further involve receiving an indication of the response signal from a sensor via the electrical circuit and transmitting the indication of the response signal to the controller via the network. The controller may be remote from the stimulator and the sensor.

### BRIEF DESCRIPTION OF THE DRAWINGS

FIG. 1 is a schematic diagram of an example system for monitoring a characteristic of a material.

FIG. 2 is a schematic diagram of another example system for monitoring a characteristic of a material, the system having a combination of stimulators and sensors.

FIG. 3 is a schematic diagram of another example system for monitoring a characteristic of a material, the system having a controller remote from a stimulator and sensor.

FIG. 4 is a schematic diagram of another example system for monitoring a characteristic of a material, the system having a stimulator and sensor incorporated into a monitoring device to monitor material in a material container.

FIG. 5 is a schematic diagram of an example monitoring device for monitoring a characteristic of a material.

FIG. 6 is a schematic diagram of an example monitoring device for monitoring a characteristic of a material, the monitoring device installed at a water faucet.

FIG. 7 is a schematic diagram of an example monitoring device for monitoring a characteristic of a material, the monitoring device installed in a wine bottle.

FIG. 8 is a schematic diagram of an example monitoring device for monitoring a characteristic of a material, the monitoring device installed in a toilet.

FIG. 9 is a schematic diagram of an example monitoring device for monitoring a characteristic of a material, the monitoring device installed in a cup.

FIG. 10 is a schematic diagram of an example monitoring device for monitoring a characteristic of a material, the monitoring device for use as a probe.

FIG. 11 is a schematic diagram of example physical patterns of stimulus signals to be applied to a material.

### DETAILED DESCRIPTION

Many materials have characteristics which change over time in ways in which are not directly measurable by analytical testing techniques. These characteristics may be monitored by stimulating the material with a stimulus signal which may include one or a combination of an electrical signal, a magnetic signal, an optical signal, and/or an acoustic signal, and measuring a response signal, which may also include one or a combination of an electrical signal, a magnetic signal, an optical signal, and/or an acoustic signal. A characteristic of the material may be determined via a machine learning model trained to recognize characteristics of the material based on the stimulus signal and the response signal.

A stimulus may produce a cross-stimuli response, whereby a signal of one type of stimuli induces a detectable response signal of another type. For example, an optical stimulus may induce an electrical response, a magnetic response, or an acoustic response from the material. Further, combinations of stimuli may produce different responses than would be expected from applying the stimuli individually. For example, a stimulus signal including an electrical signal, a magnetic signal, an optical signal, and an acoustic signal may induce a response signal in the material which is different from the response signals which would have been produced by applying an electrical signal, a magnetic signal, an optical signal, and an acoustic signal to the material individually. Using a variety of combinations of stimuli and response signals may provide a rich dataset with which particular characteristics of a material may be identified by machine learning.

An example system for monitoring a characteristic of a material may include a stimulator to provide a stimulus signal to the material. The stimulus signal may include at least one of an electrical signal, a magnetic signal, an optical signal, and an acoustic signal. The system may further include a sensor to measure a response signal from the material. The response signal may include at least one of an electrical signal, a magnetic signal, an optical signal, and an acoustic signal. At least one of the stimulus signal and the response signal may include an optical signal or an acoustic signal. The system may further include a controller in communication with the stimulator and the sensor to apply machine learning to determine a characteristic of the material based on the stimulus signal and the response signal, wherein the characteristic is not directly measurable. The machine learning may be applied via a machine learning model trained with library data of previously measured stimulus signals and response signals to recognize the characteristic of the material.

The stimulus signal may include each of an electrical signal, a magnetic signal, an optical signal, and an acoustic signal, and the response signal may include each of an electrical signal, a magnetic signal, an optical signal, and an acoustic signal. This combination of signals may induce a response signal in the material which provides a response signal which may be used to detect characteristics of the material by machine learning.

The stimulator and sensor may be incorporated into a monitoring device having a body to be installed at a material container such that the stimulator and sensor may interact with the material in the material container. The material container may include a material conduit through which the material to be analyzed may be carried, such as a pipe for liquids, a chute or conveyor for solids, a water faucet, or a toilet. The material container may include a vessel or other container to hold a material to be analyzed, such as a cup to contain a liquid, or a wine bottle to contain wine.

The controller may be remote from the stimulator and the sensor. Thus, the system may include a communication interface in communication with the stimulator and the sensor via an electrical circuit and in communication with the controller via a network. The communication interface may receive an indication of the stimulus signal from the controller via the network and transmit the indication of the stimulus signal to the stimulator via the electrical circuit. The communication interface may also receive an indication of the response signal from the sensor via the electrical circuit and transmit the indication of the response signal to the controller via the network. The characteristics of a material may thereby be monitored remotely.

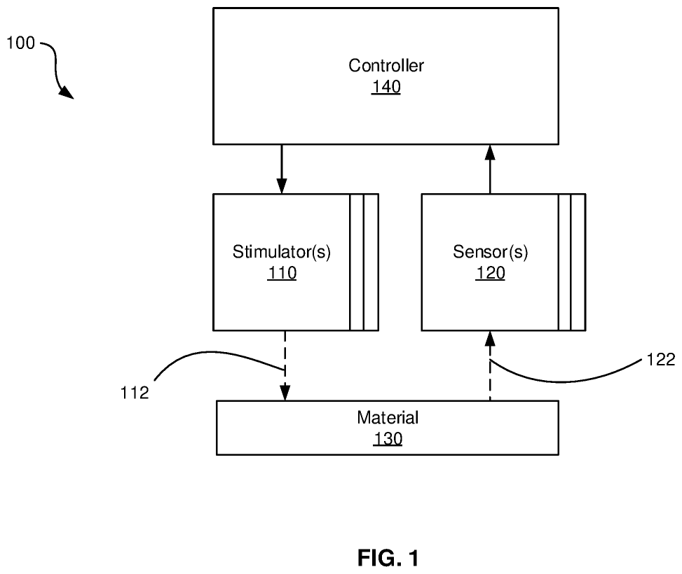

FIG. 1 is a schematic diagram of an example of such a system 100. The system 100 includes one or more stimulators 110 to provide a stimulus signal 112 to a material 130. The stimulus signal 112 includes at least one of an electrical signal, a magnetic signal, an optical signal, and an acoustic signal. The system 100 further includes one or more sensors 120 to measure a response signal 112 from the material 130. The response signal 122 includes at least one of an electrical signal, a magnetic signal, an optical signal, and an acoustic signal. Although represented as single stimulus signal 112 and response signal 122, it is to be understood that such signals 112, 122, may include one or a plurality of signals corresponding to different stimuli. At least one of the stimulus signal and the response signal may include an optical signal or an acoustic signal.

The one or more stimulators 110 may include any combination of an optical stimulator, an acoustic stimulator, an electrical stimulator, and a magnetic stimulator, to produce a signal to stimulate the material 130. For example, the one or more stimulators 110 may include an optical stimulator, and thus the stimulus signal may include an optical signal. As another example, the one or more stimulators 110 may include an acoustic stimulator, and thus the stimulus signal may include an acoustic signal.

An optical stimulator may include any device which is to emit electromagnetic radiation to the material 130 to optically stimulate the material 130. For example, an optical stimulator may include a light emitting diode (LED). Other example optical stimulators include a photocathode, an incandescent light bulb, a xenon light lamp, an argon lamp, a neon lamp, a cathode ray tube, or any other optical light stimulating device that can convert electrical energy into an optical light stimulus. An optical stimulator may emit electromagnetic radiation from any portion of the electromagnetic spectrum, such as the visible light, near infrared light, mid infrared light, far infrared light, near ultraviolet light, extreme ultraviolet light, elf, slf, ulf, vlf, lf, mf, hf, vhf, uhf, shf, ehf, soft x-rays, hard x-rays, or gamma rays.

An acoustic stimulator may include any device which is to emit sound waves to the material 130 to acoustically stimulate the material 130. For example, an acoustic stimulator may include a piezo device. Other example acoustic stimulators include an electromagnetic speaker or other devices manufactured from crystals, semiconductors or electromagnetic coils which may be used as acoustic stimulators. An acoustic stimulator may emit sound waves from the audible spectrum of sound, infrasound, or ultrasound.

An electrical stimulator may include an electrode to make electrical contact with the material 130 to allow an electrical stimuli to be transmitted to the material 130. An electrical stimulator may be an electrode made of any suitable material for electrical conductivity, including gold, a gold-plated metal, platinum, a platinum-plated metal, carbon, graphite, graphene, silver, silver chloride, silicon, germanium, tin, iron, copper, or brass, or other suitable materials.

A magnetic stimulator may include a magnetic coil to magnetically couple with the material 130 to allow it to magnetically stimulate the material 130.

Any one of an optical signal, an acoustic signal, an electrical signal, and a magnetic signal may be referred to as a stimulating signal profile when used to stimulate the material 130. A stimulating signal profile may include a static or a time-varying signal, and may include continuous, discrete, periodic, or aperiodic signals, or combinations thereof. A stimulating signal profile may include a sinusoidal oscillating signal. A stimulating signal profile may vary in one or a plurality of dimensions. Where a combination of signals is used, any one of the signals may be varied simultaneously or independently. Further, a response signal 122 may be measured simultaneously with, or after, a stimulating signal 112 is transmitted to the material 130.

Further, the one or more sensors 120 may include any combination of an optical sensor, an acoustic sensor, an electrical sensor, and a magnetic sensor, to measure the corresponding signal from the material 130. For example, the one or more sensors 120 may include an optical sensor, and thus the response signal may include an optical signal. As another example, the one or more sensors 120 may include an acoustic sensor, and thus the response signal 122 may include an acoustic signal.

An optical sensor may include any device which is to measure electromagnetic radiation emitted from the material 130 as an optical response signal. For example, an optical sensor may include a photodiode. Other example optical sensors include a charged coupled device (CCD), or any other optical light receiving device that can convert light energy into electrical energy. An optical sensor may receive electromagnetic radiation from any portion of the electromagnetic spectrum, such as the visible light, near infrared light, mid infrared light, far infrared light, near ultraviolet light, extreme ultraviolet light, elf, slf, ulf, vlf, lf, mf, hf, vhf, uhf, shf, ehf, soft x-rays, hard x-rays, or gamma rays.

An acoustic sensor may include any device which is to detect sound waves emanating from the material 130. For example, an acoustic sensor may include a piezo device. Other example acoustic sensors include an electromagnetic coil microphone, a crystal microphone, or other devices which may be used as acoustic sensors. An acoustic sensor may detect sound waves from the audible spectrum of sound, infrasound, or ultrasound.

An electrical stimulator may include an electrode to make electrical contact with the material 130 to allow an electrical measurement on the material 130 to be performed. An electrical stimulator may be made of any suitable material for electrical conductivity, including gold, a gold-plated metal, platinum, a platinum-plated metal, carbon, graphite, graphene, silver, silver chloride, silicon, germanium, tin, iron, copper, or brass, or other suitable materials. An electrode may conduct impedance spectroscopy, also known as dielectric spectroscopy, for electrically stimulating the material 130 and performing a measurement on the material 130. It is to be understood, however, that in other examples, other electro-analytical methodologies can be performed, such as potentiometry, coulometry, voltammetry, square wave voltammetry, stair-case voltammetry, cyclic voltammetry, alternating current voltammetry, amperometry, pulsed amperometry, galvanometry, and polarography, and other suitable electro-analytical methodologies.

A magnetic sensor may include a magnetic coil to magnetically couple with the material 130 to allow a magnetic measurement from the material 130 to be performed.

In any case, it is to be understood that any stimulator or sensor may include an electronic device, and thus an indication of the stimulus signal 112 may be received by the one or more stimulators 110 as an electrical signal and converted by the relevant stimulator 110 into the corresponding optical, acoustic, electrical, or magnetic signal to be provided to the material 130. Similarly, it is to be understood that a sensor 120 may measure a response signal 122 and convert the measurement into an electrical signal as an indication of the response signal 122 to be transmitted from the sensor 120 to the controller 140.

Various combinations of stimulus signals and response signals are contemplated, and these stimulus signals and response signals may give rise to cross-stimuli interactions. As an example, the stimulus signal 112 may include an optical signal, and the response signal 122 may be particularly pronounced in the electrical signal band. As another example, the stimulus signal may include an acoustic signal, and the response signal 122 may be particularly pronounced in the magnetic signal band. As another example, the stimulus signal 112 may include an electrical signal, a magnetic signal, an optical signal, and an acoustic signal, and the response signal 122 may be particularly pronounced in the electrical signal and a magnetic signal bands. As another example, the stimulus signal 12 may include an electrical signal, a magnetic signal, an optical signal, and an acoustic signal, and the response signal 122 may be particularly pronounced in the electrical signal, magnetic signal, and acoustic signal bands.

An optical stimulator and sensor may be configured to be of a variety of different shapes, such as cylinders, rectangular prisms, spheres, hemispheres, pyramids, or any other suitable shape to emit or receive light from the material 130. The shape of the optical stimulator and sensor may impact the optical signal or optical response and thus may provide additional variables for the machine learning model which to facilitate the monitoring of characteristics of the material 130.

The system 100 further includes a controller 140 in communication with the one or more stimulators 110 and the one or more sensors 120. The controller 140 may include a processor, a microcontroller, a state machine, a logic gate array, an application-specific integrated circuit (ASIC), a system-on-a-chip (SOC), a field-programmable gate array (FPGA), or similar, capable of executing, whether by software, hardware, firmware, or a combination of such or similar, the techniques described herein. The controller 140 may include a computing device such as a smartphone, a notebook computer, a desktop computer, a server, one or more cloud computing devices, or a combination of such or similar, having processing, storage, and communication means.

The controller 140 is to apply machine learning to determine a characteristic of the material based on the stimulus signal 112 and the response signal 122, wherein the characteristic is not directly measurable. The machine learning is applied via a machine learning model trained with library data including previously measured stimulus signals and response signals to recognize the characteristic of the material. Thus, the controller 140 may store measurement data which includes the measured response signals 122 and the correspondingly used stimulus signals 112. The controller 140 may further store library data which includes previously measured response signals 122 and previously used stimulus signals 112 correlated to (or otherwise related to) characteristics of materials. The library data may include data relating to known optical properties, acoustic properties, electrical properties, or magnetic properties of materials. The measurement data may be contributed to library data for further training of the machine learning model.

Some characteristics, although not directly measurable, may be recognized by a machine learning model based on such measurement data and library data. The library data may relate measured optical, acoustic, electrical, and magnetic properties of a material to known, not directly measurable, characteristics of the material. A machine learning model may be trained to recognize that a measurement of a particular magnetic signal profile emanating from water flowing through a water faucet, following a particular electrical stimulus, indicates the presence of a contaminant in the water. The presence of such a contaminant may be not directly measurable in that its detection may involve a length laboratory analysis which is not useful to the user of the water faucet who may benefit from a more immediate analysis. The library data may include other known relationships, such as that the detection of a particular optical signal profile indicates the presence of a particular nutrient in a particular foodstuff, or that a particular combination of acoustic signal profile and magnetic signal profile emanating from an organism after a particular electrical stimulant indicates that the organism contains a certain quantity of chemical.

The machine learning model may apply any one of various machine learning techniques to determine a characteristic of the material 130. For example, a neural network algorithm that employs a Bayesian algorithm and a decision tree analysis to classify a response signal 122 and report the classified result in order to classify the characteristics of the material 130 may be employed. In other examples, principal component analysis (PCA) may be used on a response signal 122 to report on the status of the material 130 and also classify its characteristics. In other examples, principal component regression may be used on a response signal 122 to report on the status of the material 130 and also classify its characteristics. In other examples, other suitable data analysis techniques may be used, such as linear regression, polynomial regression, clustering analysis, correlation, neural network machine learning algorithms, support vector machine algorithms, random forest algorithms, convolution neural network algorithms, deep learning, deep belief networks, deep QA networks, or other appropriate algorithms. Machine learning algorithms may include supervised machine learning algorithms or unsupervised machine learning algorithms.

The machine learning model may comprise several distinct machine learning algorithms. For example, a first machine learning algorithm may determine whether the material 130 contains E. Coli, a second machine learning algorithm may determine whether the material 130 contains a particular chemical analyte, and so on.

Prior to running through a machine learning model, a response signal 122 may undergo a mathematical transformation. The mathematical transform that may include a Fourier transform, Fast Fourier Transform (FFT), Discrete Fourier Transform (DFT), Laplace transform, Z transform, Hilbert transform, Discrete Cosine transform, wavelet transform, discrete wavelet transform, Infinite Impulse Response (IIR), Finite Impulse Response (FIR), or their discrete or accelerated variants, or other mathematical transforms. The mathematical transform can be made in any possible domain such, as but not limited to, time and space domain, frequency domain, Z-plane analysis (Z-domain), and Wavelet analysis, and any such relevant domain or analysis methodology.

Thus, the system 100 may be used to perform a method for monitoring a characteristic of a material. The method involves providing a stimulus signal to a material, the stimulus signal including at least one of an electrical signal, a magnetic signal, an optical signal, and an acoustic signal. The method further involves measuring a response signal from the material, the response signal including at least one of an electrical signal, a magnetic signal, an optical signal, and an acoustic signal. At least one of the stimulus signal and the response signal includes an optical signal or an acoustic signal. The method further involves determining a characteristic of the material based on the stimulus signal and the response signal, wherein the characteristic is not directly measurable, by applying machine learning via a machine learning model trained with library data of previously measured stimulus signals and response signals to recognize the characteristic of the material. The method may be instantiated in a non-transitory computer-readable medium which, when executed, causes a processor of a computing device to perform the method.

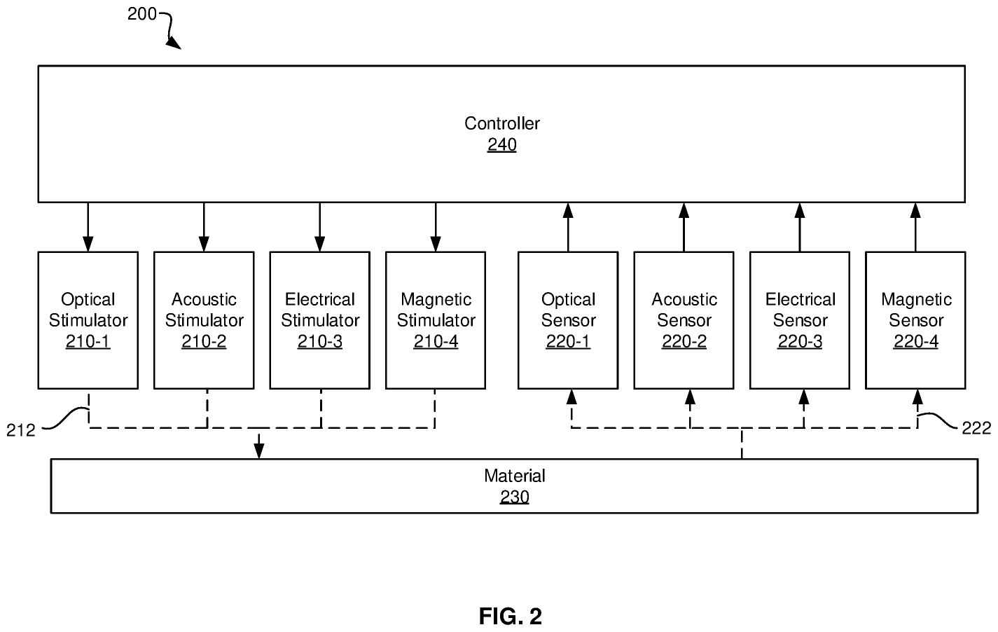

FIG. 2 is a schematic diagram showing another example system 200 for monitoring a characteristic of a material. The system 200 may be similar to the system 100, with analogous elements being labelled in the “200” series rather than the “100” series, and thus includes stimulators 210 to produce a stimulus signal 212, sensors 220 to measure a response signal 222, a material 230, and a controller 240. For further description of the above elements, the description of the system 100 of FIG. 1 may be referenced.

The system 200 includes an optical stimulator 210-1, an acoustic stimulator 210-2, an electrical stimulator 210-3, and a magnetic stimulator 210-4. Further, the system 200 includes an optical sensor 220-1, an acoustic sensor 220-2, an electrical sensor 220-3, and a magnetic sensor 220-4. Each of the stimulators are to be used such that the stimulus signal 212 includes an electrical signal, a magnetic signal, an optical signal, and an acoustic signal. Further, each of the sensors are to be used such that the response signal 222 includes an electrical signal, a magnetic signal, an optical signal, and an acoustic signal. This combination of signals may induce a response signal 222 in the material 230 which is different from the response signal which would have been produced by applying the various stimulus signals individually. The response signal generated from the combined stimuli may allow for the detection of particular characteristics of the material 230 by machine learning.

Thus, the system 200 may be used to perform a method for monitoring a characteristic of a material. The method involves providing a stimulus signal to a material, the stimulus signal including an electrical signal, a magnetic signal, an optical signal, and an acoustic signal. The method further involves measuring a response signal from the material, the response signal including an electrical signal, a magnetic signal, an optical signal, and an acoustic signal. The method further involves determining a characteristic of the material based on the stimulus signal and the response signal, wherein the characteristic is not directly measurable, by applying machine learning via a machine learning model trained with library data of previously measured stimulus signals and response signals to recognize the characteristic of the material. The method may be instantiated in a non-transitory computer-readable medium which, when executed, causes a processor of a computing device to perform the method.

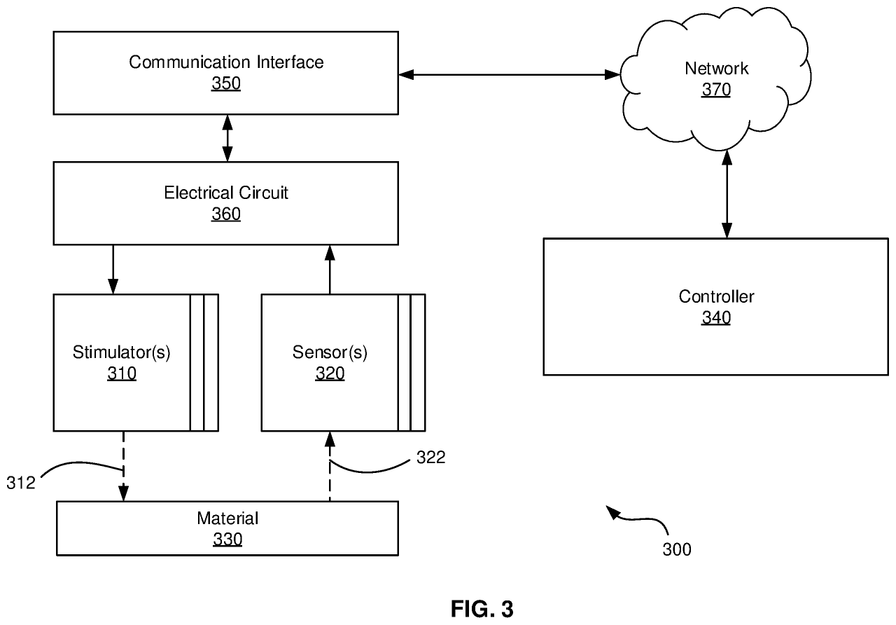

FIG. 3 is a schematic diagram showing another example system 300 for monitoring a characteristic of a material. The system 300 may be similar to the system 100, with analogous elements being labelled in the “300” series rather than the “100” series, and thus includes one or more stimulators 310 to produce a stimulus signal 312, one or more sensors 320 to measure a response signal 322, a material 330, and a controller 340. For further description of the above elements, the description of the system 100 of FIG. 1 may be referenced.

In system 300, the controller 340 is remote from the one or more stimulators 310 and the one or more sensors 320. The system 300 further includes a communication interface 350 in communication with the one or more stimulators 310 and the one or more sensors 320 via an electrical circuit 360.

The communication interface 350 is in communication with the controller 340 via a network 370. The communication interface 350 is to receive an indication of the stimulus signal 312 from the controller 340 via the network 370 and transmit the indication of the stimulus signal 312 to the one or more stimulators 310 via the electrical circuit 360. The communication interface 350 is also to receive an indication of the response signal 322 from the one or more sensors 320 via the electrical circuit 360 and transmit the indication of the response signal 322 to the controller 340 via the network 370. Thus, the communication interface 350 acts as a data router between the one or more stimulators 310 and sensors 320 and the controller 340.

The network 370 may include a wireless cellular data network, a Wi-Fi network, a local-area network, a wide-area network (WAN), a Bluetooth pairing or connection, the internet, a virtual private network (VPN), a combination of such, and similar. Further, the communication interface 350 may include a wireless device such as a smartphone, notebook computer, or other computing device. The communication interface 350 may also include an antenna or other suitable communication device.

The controller 340 may be in communication with other parties via the network 370, such as consumers, owners, retailers, or manufacturers who may have an interest in the status of the material 330.

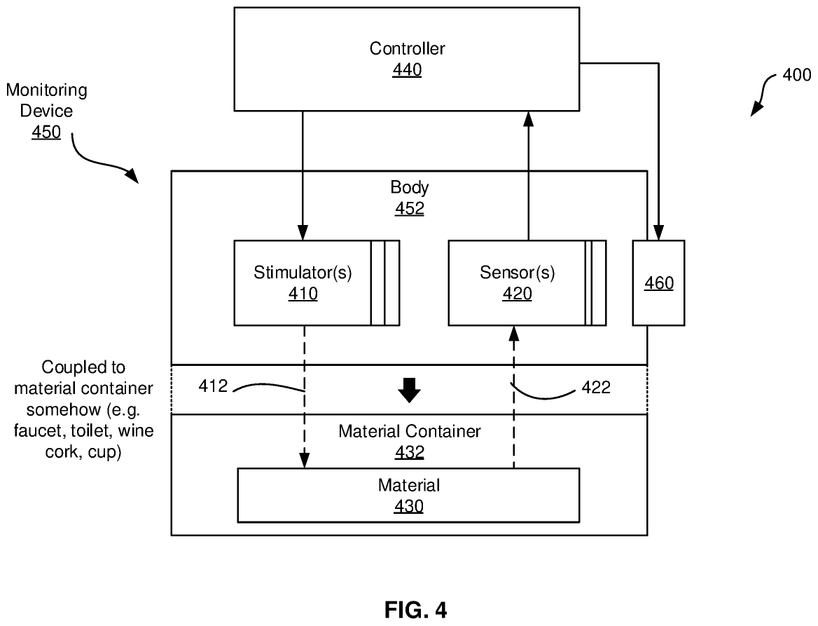

FIG. 4 is a schematic diagram showing another example system 400 for monitoring a characteristic of a material. The system 400 may be similar to the system 100, with analogous elements being labelled in the “400” series rather than the “100” series, and thus includes one or more stimulators 410 to produce a stimulus signal 412, one or more sensors 420 to measure a response signal 422, a material 430, and a controller 440. For further description of the above elements, the description of the system 100 of FIG. 1 may be referenced.

In system 400, the material 430 is in a material container 432, and the one or more stimulators 410 and the one or more sensors 420 are incorporated into a monitoring device 450. The monitoring device 250 includes a body 452 to be installed at the material container 432 such that the one or more stimulators 410 and the one or more sensors 420 may interact with the material 430 in the material container 432. The monitoring device 450 may include a power supply such as a wired power connection, a battery, a solar cell, an energy harvester, or other suitable power supply. An energy harvester may harvest energy from a communication field, movement of the material 430, heat from the material 430, or another source.

In the example shown, the controller 440 is outside the body 452, and in such examples, the controller 440 may communicate with the one or more stimulators 410 and the one or more sensors 420 remotely via a network, communication interface, and electrical circuit, similar to as described in system 300 of FIG. 3. In other examples, the controller 440 may be incorporated within the body 452 and thus a part of the monitoring device 450. In still other examples, the controller 440 may be outside the body 452 but in communication with the one or more stimulators 410 and the one or more sensors 420 in any suitable way, such as through an electrical connection.

The material container 432 may include a material conduit through which the material to be analyzed is carried, such as a pipe for liquids, a chute or conveyor for solids, a water faucet, or a toilet. The material container 432 may include a vessel or other container to hold a material to be analyzed, such as a cup to contain urine to be analyzed, or a wine bottle to contain a wine to be analyzed.

The body 452 may be made of any material suitable and in any suitable shape for the application. In the example of a wine bottle, the body 452 may be shaped to fit into a wine bottle cork, or as a wine bottle cork itself, and may be made of plastic or cork material.

The monitoring device 450 may include an alert device 460 to indicate the identification of a particular characteristics of the material 430. The alert device 460 may include a simple single-color LED, a multi-color LED, a moving coil galvanometer (voltmeter or current meter, or any suitable meter), a piezoelectric transducer, or speaker, or buzzer, or siren, or relay switch, or an optical bar graph, a counter (such as a numerical counter or any suitable counter), an LCD display, or any other indicator.

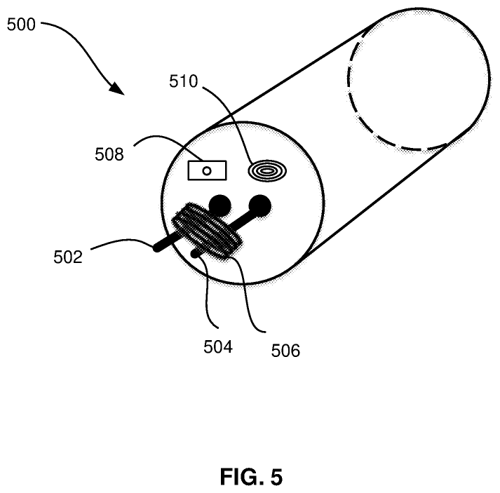

FIG. 5 is a schematic diagram of an example monitoring device 500 for monitoring a characteristic of a material. The monitoring device 500 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The monitoring device 500 includes an electrical stimulator 502, an electrical sensor 504, a magnetic stimulator 506, an optical stimulator 508, and an acoustic stimulator 510. Thus, the monitoring device 500 may stimulate a material with an optical signal, an acoustic signal, an electrical signal, and an acoustic signal, and may measure an electrical response signal.

In other examples, a monitoring device may include an optical stimulator, an optical sensor, a stimulating electrode, a sensing electrode, and a magnetic coil to act as a stimulator or a sensor.

In other examples, a monitoring device may include a stimulating electrode, a sensing electrode, and a reference electrode to conduct electro-chemical analysis, a magnetic coil to act as a stimulator or a sensor, an optical sensor, and an acoustic sensor.

In other examples, a monitoring device may include an optical stimulator, an acoustic stimulator, a stimulating electrode, a sensing electrode, and a magnetic coil to act as a stimulator or a sensor.

In other examples, a monitoring device may include an acoustic sensor, an electrical sensor, and a magnetic coil to act as a stimulator or a sensor.

In other examples, a monitoring device may include an optical stimulator, an electrical stimulator, an electrical sensor, and a magnetic coil to act as a stimulator or a sensor.

In other examples, a monitoring device may include an optical stimulator, an electrical sensor, and a magnetic coil to act as a stimulator or a sensor.

In other examples, a monitoring device may include an electrical sensor, a magnetic coil to act as a sensor, and an optical stimulator.

In other examples, a monitoring device may include an electrical sensor, a magnetic coil to act as a sensor, and an acoustic stimulator.

In other examples, a monitoring device may include both an optical stimulator and an acoustic stimulator.

In other examples, a monitoring device may include a stimulating electrode, a sensing electrode, and a reference electrode to conduct electro-chemical analysis, a magnetic coil as a sensor, and one or both of an optical stimulator and an acoustic stimulator.

In other examples, a monitoring device may include a stimulating electrode, a sensing electrode, and a reference electrode to conduct electro-chemical analysis, a magnetic coil as a sensor, an optical stimulator, an acoustic stimulator, an one or both of an optical sensor and an acoustic sensor.

In other examples, a monitoring device may include an electrical sensor, one or both of an optical sensor and an acoustic sensor, and a magnetic coil as a stimulator and/or a sensor.

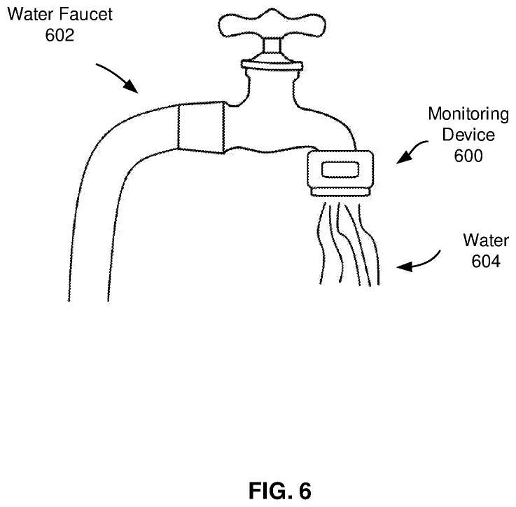

FIG. 6 is a schematic diagram of an example monitoring device 600 installed at a water faucet 602 to monitor a characteristic of water 604. The monitoring device 600 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The body of the monitoring device 600 may be configured to be installed at the outlet of the water faucet 602, or may be configured to be installed in piping upstream of the outlet. The monitoring device 600 may thereby monitor water 604 as it passes through the water faucet 602.

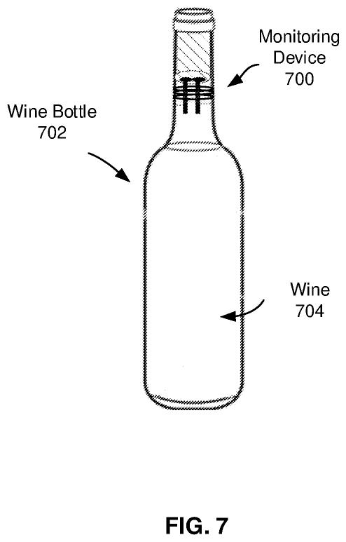

FIG. 7 is a schematic diagram of an example monitoring device 700 installed in a wine bottle 702 to monitor a characteristic of wine 704. The monitoring device 700 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The body of the monitoring device 700 may be incorporated into a wine bottle cork, and may be configured to be installed at the outlet of the wine bottle 702. The monitoring device 700 may thereby monitor wine 704 as it ages inside the wine bottle 702. Thus, in some applications, it may be determined whether the wine 704 is within an optimal taste window versus outside an optimal taste window. In other applications, it may be determined whether the wine 704 is sufficiently fermented and it is now ready to ship to market. Particular taste characteristics of the wine 704 may be monitored, such as its sweetness of flavor, acidity, tannin, fruitiness of flavor, body, aroma, or any other characteristic of the wine 704 which is not directly measurable.

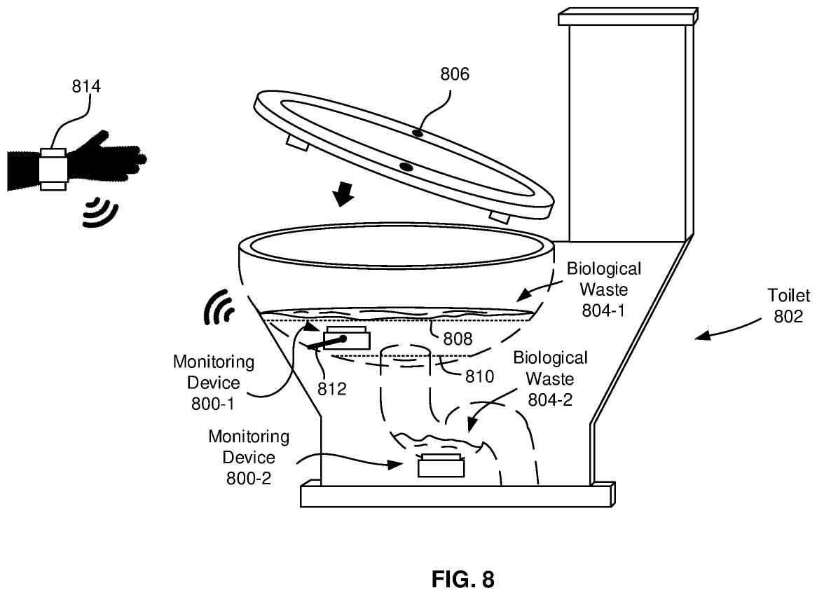

FIG. 8 is a schematic diagram of an example monitoring device 800 installed in a toilet 802 to monitor a characteristic of biological waste 804. The monitoring device 800 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The body of the monitoring device 800 may be configured to be installed in the bowl of the toilet 802, or in piping downstream of the toilet bowl. Thus, a monitoring device 800-1 is shown to monitor biological waste 804-1 in the bowl of the toilet 802, and a monitoring device 800-2 is shown to monitor biological waste 804-2 in piping downstream of the bowl of the toilet 802. The monitoring device 800 may thereby monitor biological waste 804 as it arrives in or passes through the toilet 802.

A monitoring device 800 thereby provides a way to monitor one's health through monitoring one's biological waste such as urine and feces in a regular routine manner. Such monitoring may provide valuable health-related data which an individual may provide to one's physician. Further, such monitoring may provide an early warning mechanism, whereby the detection of a particular analyte in the biological waste may trigger an alert which prompts the individual to seek further medical attention. Further, with the monitoring device 800 being installed at a toilet, such monitoring may take place from the comfort of one's own home. Moreover, the data gathered from the monitoring device 800 may be used to generate meal plans or other corrective actions for an individual to improve the individual's health.

Example health-related data to be gathered from the biological waste 804 may include the presence of specific analytes, such as prostate specific antigen (PSA) in urine to track and alert for prostate cancer. Another example of health-related data is the presence of bacteria, such as E. Coli, or coliforms to, for example, provide an alert for the possibility of a urinary tract infection (UTI). In other examples, the deficiency of iron, potassium, or other minerals may be monitored. Data analytics and machine learning techniques such as those described above may be used to examine the health of a user. For example, a canonical correlation may be used to report on the status of the health of the user, i.e., whether it is within the user's optimal health window or approaching its an sub-optimal point, a disease or illness point, or the user's expiry point, and estimating how much time may be left before the user reaches his or her expiry point. Further, such health-related data may be used to monitor the effectiveness of medications used by a user, for titrating medications, or for developing a closed loop metrology approach for particular medications that could be harmful outside of a very specific dosing range.

The monitoring device 800 may be connected to seat sensors 806 which may detect the presence of a user of the toilet 802 to cause the monitoring device 800 to initiate testing procedures. The monitoring device 800 may be shut off to conserve power until the seat sensors 806 detect a user, at which time the monitoring device 800 may be switched on and begin providing stimulants and taking measurements.

Further, in some examples, a monitoring device 800 may be installed in a toilet seat directly to make direct contact with the user's skin to determine characteristics of the user.

In some examples, the toilet 802 may include a fecal trap door 808 which may separate fecal material from other undesired materials such as toilet paper in the bowl of the toilet 802. The toilet 802 may include a urine pass-through membrane 810 which may allow urine material to pass through it to separate the urine material from the fecal material. Thus, the monitoring device 800-1 may be positioned to monitor fecal material whereas the monitoring device 800-2 may be positioned to monitor urine material. The monitoring device 800-1 may be connected to an actuator arm 812 which may move the monitoring device 800-1 to make contact with fecal material in the bowl of the toilet 802.

In some examples, the user of the toilet 802 may carry a communication device such as a wearable device 814 which communicates with a monitoring device 800 to identify the user to the monitoring device 800 and to gather and store measurement data from the monitoring device 800. Thus, multiple users of the same toilet 802 may independently gather and track health-related data. The wearable device 814 may include a radio frequency identification (RFID) wrist band, a smart phone, or other portable device. Communication between the portable device and the monitoring device 800 may be encrypted for secure information transfer.

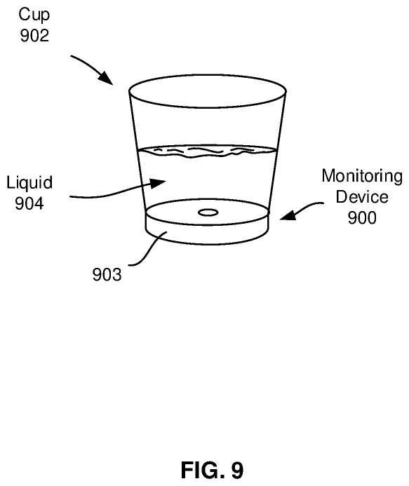

FIG. 9 is a schematic diagram of an example monitoring device 900 installed in a cup 902 to monitor a characteristic of a liquid 904. The monitoring device 900 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The body of the monitoring device 900 may be configured to be installed in a base portion 903 of the cup 902, or in side walls of the cup 902. The monitoring device 900 may thereby monitor the liquid 904 as it rests in the cup 902.

In some examples, the base portion 903 containing the monitoring device 900 may be detachable from the cup 902 for maintenance, replacement, or cleaning purposes. In such examples, the monitoring device 900 may be used to monitor biological waste, such as urine.

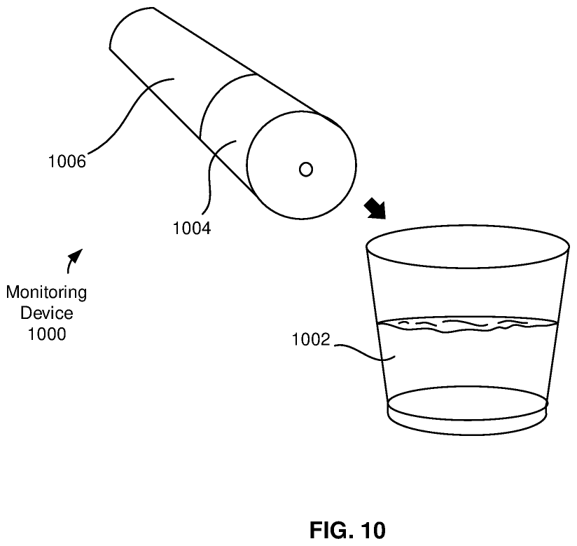

FIG. 10 is a schematic diagram of an example monitoring device 1000 to be used as a probe to monitor a characteristic of a material 1002. The monitoring device 1000 may be similar to the monitoring device 450 of FIG. 4, and thus may include one or more stimulators, one or more sensors, a body, and in some examples a controller. For further description of the above elements, the description of the monitoring device 450 of FIG. 4 may be referenced.

The monitoring device 1000 may include a detachable head portion 1004 which is detachable from a base portion 1006 for sanitary purposes. The monitoring device 1000 may be used to contact a material 1002 and make measurements of the material 1002, such as biological waste. For example, the head portion 1004 of the monitoring device 1000 may be inserted into a cup filled with urine, or directly into a volume of feces. The head portion 1004 may be removed for cleaning or replaced.

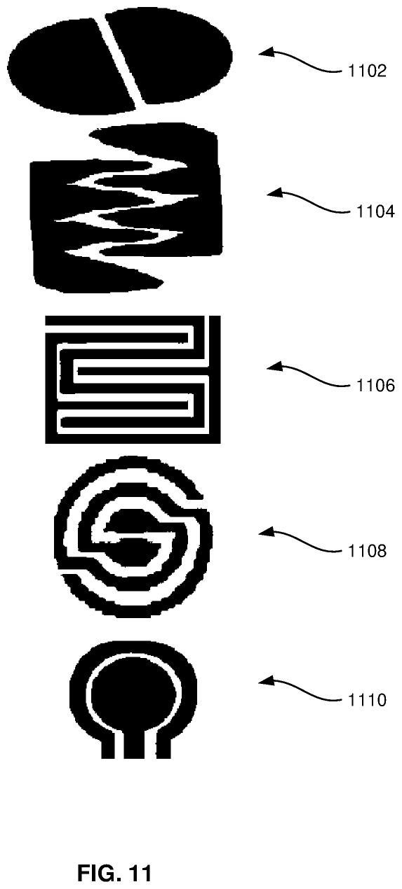

FIG. 11 is a schematic diagram of example physical patterns of stimulus signals to be applied to a material. A stimulus signal may take the form of a particular physical pattern to induce a particular response signal from the material. Different physical patterns may induce different responses from the material. Example physical patterns include patterns 1102, 1104, 1106, 1108, and 1110. A stimulus may be directed along the paths of the patterns 1102, 1104, 1106, 1108, or other paths, to induce a particular response. For example, an electrode may be translated or rotated to electrically stimulate a material along a path of pattern 1102, 1104, 1106, 1108, or 110.

Thus, a characteristic of a material which is not directly measurable may be monitored by stimulating the material with a stimulus signal and measuring a response signal. The stimulus and the response may include one or more of an optical signal, an acoustic signal, an electrical signal, and a magnetic signal. A characteristic of the material may be determined via a machine learning model trained to recognize characteristics of the material based on the stimulus signal and the response signal. A stimulus may produce a cross-stimuli response, whereby a signal of one type of stimuli induces a detectable response signal of another type. Using a variety of combinations of stimuli and response signals may provide a rich dataset with which particular characteristics of a material may be identified by machine learning. The stimulators and sensors may be incorporated into a monitoring device h to be installed at a material container such as a water faucet, wine bottle, cup, or toilet, such that the stimulator and sensor may interact with the material in the material container. A controller may be remote from the stimulator and the sensor to allow for remote monitoring. Therefore, products and biological materials may be reliably monitored to gather insights which may otherwise not be directly attainable.

Additional examples of the systems and devices described herein are contemplated. For example, although the systems and devices described herein are described with respect to applications in monitoring water, wine, biological waste, and cups, it is to be understood that other materials may be monitored, such as other foodstuffs or beverages, medications, vaccines, solids, gases, plasma, chemicals, chemical reactions, or any other product of interest, or for monitoring bodily fluids other than biological waste, or for monitoring biological organisms such as cells.

The scope of the claims should not be limited by the above examples, but should be given the broadest interpretation consistent with the description as a whole.

1. A system for monitoring a characteristic of a material, the system comprising:
a stimulator to provide a stimulus signal to the material, the stimulus signal including at least one of an electrical signal, a magnetic signal, an optical signal, and an acoustic signal;
a sensor to measure a response signal from the material, the response signal including at least one of an electrical signal, a magnetic signal, an optical signal, and an acoustic signal, wherein at least one of the stimulus signal and the response signal includes an optical signal; and
a controller in communication with the stimulator and the sensor, the controller to apply machine learning to determine a characteristic of the material based on the stimulus signal and the response signal, wherein the characteristic is not directly measurable, and wherein the machine learning is applied via a machine learning model trained with library data of previously measured stimulus signals and response signals to recognize the characteristic of the material.

2. The system of claim 1, wherein the stimulator includes an optical stimulator and the stimulus signal includes an optical signal.

3. The system of claim 2, wherein the optical stimulator includes a light emitting diode (LED).

4. The system of claim 2, wherein the response signal includes a magnetic signal.

5. The system of claim 1, wherein the stimulus signal includes an electrical signal, a magnetic signal, an optical signal, and an acoustic signal, and wherein the response signal includes an electrical signal and a magnetic signal.

6. The system of claim 1, wherein the sensor includes an optical sensor, the response signal includes an optical signal, and the optical sensor includes a photodiode.

7. The system of claim 1, wherein the stimulator and the sensor are incorporated into a device for installation at a toilet, and wherein the material comprises biological waste.

8. A system for monitoring a characteristic of a material, the system comprising:
a stimulator to provide a stimulus signal to the material, the stimulus signal including at least one of an electrical signal, a magnetic signal, an optical signal, and an acoustic signal;
a sensor to measure a response signal from the material, the response signal including at least one of an electrical signal, a magnetic signal, an optical signal, and an acoustic signal, wherein at least one of the stimulus signal and the response signal includes an acoustic signal; and
a controller in communication with the stimulator and the sensor, the controller to apply machine learning to determine a characteristic of the material based on the stimulus signal and the response signal, wherein the characteristic is not directly measurable, and wherein the machine learning is applied via a machine learning model trained with library data of previously measured stimulus signals and response signals to recognize the characteristic of the material.

9. The system of claim 8, wherein the stimulator includes an acoustic stimulator and the stimulus signal includes an acoustic signal.

10. The system of claim 9, wherein the acoustic stimulator includes a piezo device.

11. The system of claim 8, wherein the stimulus signal includes an electrical signal, a magnetic signal, an optical signal, and an acoustic signal, and wherein the response signal includes an electrical signal, a magnetic signal, and an acoustic signal.

12. The system of claim 8, wherein the sensor includes an acoustic sensor, the response signal includes an acoustic signal, and the acoustic sensor includes a piezo device.

13. The system of claim 8, wherein the stimulator and the sensor are incorporated into a device for installation at a toilet, and wherein the material comprises biological waste.

14. A system for monitoring a characteristic of a material, the system comprising:
a stimulator to provide a stimulus signal to the material, the stimulus signal including an electrical signal, a magnetic signal, an optical signal, and an acoustic signal;
a sensor to measure a response signal from the material, the response signal including an electrical signal, a magnetic signal, an optical signal, and an acoustic signal; and
a controller in communication with the stimulator and the sensor, the controller to apply machine learning to determine a characteristic of the material based on the stimulus signal and the response signal, wherein the characteristic is not directly measurable, and wherein the machine learning is applied via a machine learning model trained with library data of previously measured stimulus signals and response signals to recognize the characteristic of the material.

15. The system of claim 14, wherein the controller is remote from the stimulator and the sensor, and wherein the system further comprises:
a communication interface in communication with the stimulator and the sensor via an electrical circuit and in communication with the controller via a network, the communication interface to receive an indication of the stimulus signal from the controller via the network and transmit the indication of the stimulus signal to the stimulator via the electrical circuit, the communication interface to receive an indication of the response signal from the sensor via the electrical circuit and transmit the indication of the response signal to the controller via the network.
